(19) United States
(12) Patent Application Publication
(10) Pub. No.: US 2025/0009867 A1
(43) Pub. Date: Jan. 9, 2025

(54) COXSACKIEVIRUS B4 STRAIN AND APPLICATION THEREOF

(71) Applicant: Institute of Medical Biology, Chinese Academy of Medical Sciences, Kunming (CN)

(72) Inventors: Shaohui Ma, Kunming (CN); Changzeng Feng, Kunming (CN); Ming Zhang, Kunming (CN); Wei Guo, Kunming (CN); Yuhan Liu, Kunming (CN); Xiaohui Wang, Kunming (CN); Zhaoqing Yang, Kunming (CN); Zhanlong He, Kunming (CN); Hao Sun, Kunming (CN); Yousong Ye, Kunming (CN)

(21) Appl. No.: 18/792,611

(22) Filed: Aug. 2, 2024

(30) Foreign Application Priority Data
Aug. 2, 2023 (CN) 202310962457.X

(51) Int. Cl. A61K 39/125; A61K 39/00; A61P 31/14; C07K 16/10; C12N 7/00; C12N 15/86

(52) U.S. Cl. CPC A61K 39/125; A61P 31/14; C07K 16/1009; C12N 7/00; C12N 15/86; A61K 2039/5252; C12N 2770/32021; C12N 2770/32034; C12N 2770/32043; C12N 2770/32051; C12N 2770/32062; C12N 2770/32064

(57) ABSTRACT

A coxsackievirus B4 strain and application thereof are disclosed. The strain is named KM140-G01 and is preserved in China Center for Type Culture Collection (CCTCC) on Jun. 18, 2023, and the preservation number is CCTCC NO: V202356, the preservation address is Wuhan University, China. The strain is a humanized coxsackievirus B4 wild type single purified strain, and has strong virus replication capacity, good genetic stability and immunogenicity; the strain can be applied to the development of human coxsackievirus B4 vaccine production strains, and is suitable for the development of attenuated coxsackievirus B4 vaccine and inactivated coxsackievirus B4 vaccine; the method can also be used for establishing an infection model on Vero cells and suckling mice, and is used for CVB4 infection and pathogenic mechanism research, CVB4 vaccine evaluation and antiviral drug screening.

### CROSS REFERENCE TO RELATED APPLICATIONS

This patent application claims the benefit and priority of Chinese Patent Application No.202310962457X filed on Aug. 2, 2023, the disclosure of which is incorporated by reference herein in its entirety as part of the present application.

### SEQUENCE LISTING

The sequence listing is submitted as a XML file filed via EFS-Web, with a file name of “Substitute_Sequence_Listing. XML”, a creation date of Sep. 17, 2024, and a size of 27,373 bytes. The sequence Listing filed via EFS-Web is a part of the specification and is incorporated in its entirety by reference herein.

### TECHNICAL FIELD

The disclosure relates to the technical field of biological medicine, and more specifically, to a coxsackievirus B4 strain and application thereof.

### BACKGROUND

Coxsackievirus group B4 (CVB4) belongs to the enterovirus genus of the family picornaviridae and is a major member of the coxsackievirus group B (CVB). CVB4 infection causes a number of different degrees of disease or symptoms, such as hand-foot-and-mouth disease (HFMD), encephalitis, myocarditis, aseptic meningitis, and even death. The risk of infection in newborns and children is high in infection with CVB4, which can cause acute myocarditis and meningitis in newborns, also there is a high risk of mortality, and infection with CVB4 during pregnancy can lead to birth of infants with congenital heart lesions. In addition, CVB4 is also closely associated with the type 1 diabetes (T1 DM). Clinical studies have found that exposure to enterovirus infection in utero and childhood induces islet beta cell damage and develops into T1D in persistent infections. CVB4 has long continued to spread epidemic worldwide. Enterovirus detection reports in the US 1970-2005 indicated that CVB4 accounts for 4.2% of known serotypes and has been present in the 15 most common enteroviruses. CVB4-related reports are also available in a number of countries such as Poland, France, Italy, etc. Based on the prior arts, CVB4 cases are mostly sporadic cases in mainland China. Given that current national monitoring of HFMD is still mainly performed for few serotypes of Enterovirus type 71 (EV71), coxsackievirus type A16 (CVA16), coxsackievirus type A6 (CVA6), coxsackievirus type A10 (CVA10), and other serotypes including CVB4 are likely to be underestimated in domestic epidemic situations. As one of the most economical and effective measures for preventing diseases, it is necessary to develop a monovalent or multivalent prophylactic vaccine against CVB4 or a vaccine which includes an antigen component of CVB4 as soon as possible.

Up to date, no vaccine or specific antiviral drug against CVB4 has been approved for sale. Currently, in the field of enteroviruses, only attenuated and inactivated vaccines against polio virus and inactivated vaccines against enterovirus type 71 are used on the market. However, the above vaccine does not have cross-protection against enteroviruses of serotypes other than this serotype. Currently, vaccine studies against group B coxsackieviruses are being studied mainly by foreign researchers and institutions. Back in the '90s, See et al. from the University of California, tested the efficacy of a multivalent vaccine developed from the formalin inactivated coxsachievirus B1-6 prototype strain in mice, which vaccine reduced the severity of acute infection of clinical strains of group B coxsackieviruses In recent years, Swedish scholar Stone et al. have carried out development and preclinical testing of novel experimental hexavalent CVB vaccines, and the vaccines have better capability of inducing neutralizing antibodies in mouse and non-human primate models of several strains. In addition, the vaccine can prevent acute CVB infection, block cardiac CVB infection, and prevent CVB-induced pancreatitis and diabetes. The current research of this hexavalent CVB vaccine continues to be driven by Vactech and Provention Bio Inc., and is aimed at preventing acute CVB infection and its complications, as well as potentially delaying or preventing CVB-related autoimmune diseases TID and cellac disease.

Although various new vaccine development technical routes are developed in recent years along with the development of relevant subjects such as virology, immunology and the like, the research and the understanding of enterovirus biological characteristics and antigen epitopes are not deep enough, and the related vaccine development is mainly performed on the basis of whole virus particles. The related enterovirus vaccines (such as poliovirus vaccine and enterovirus 71 vaccine) which are used on the market are developed and produced by adopting traditional vaccine technologies such as attenuation or inactivation, and extremely remarkable immune protection effect is obtained. The products under development are also based on inactivated or attenuated vaccines. In addition, compared to attenuated live vaccines, inactivated vaccines do not have the disadvantages of virulence reversion and the risk of causing vaccine related diseases. They have good immunogenicity and safety, and are currently the most feasible and reasonable technical route. The CVB4 viruses exhibit genetic diversity, and although there is only one serotype, it has already differentiated into five genotypes. The litemiceure reports that there are differences in neutralizing antibody levels between different genotypes. Therefore, development of a CVB4 inactivated vaccine with high-efficiency protection effect on Chinese CVB4 epidemic strains (D and E genotypes are dominant genotypes) is impemiceive. It is crucial to screen strains with strong replication ability, high genetic stability, good immunogenicity, and safety in the cell matrix as vaccine production strains in the development of traditional technology routes.

Therefore, there is an urgent need for providing a CVB4 strain meeting the vaccine development requirement and applying the CVB strain to the preparation of coxsachievirus vaccine in the field.

### SUMMARY

In view of the above, the disclosure provides a coxsackievirus B4 strain which is applied to preparing coxsackievirus B4 vaccine and has good replication capacity, genetic stability and immunogenicity.

The disclosure adopts the following technical scheme:
a coxsackievirus group B4 strain having a genomic sequence as set forth in SEQ ID NO: 1 or the complete complement of the SEQ ID NO: 1 as shown.

Preferably, the strain is named as Coxsackievirus B4 KM140-G01 and its taxonomic name is Coxsackievirus B4 CVB4 KM140-G01 and is preserved in China Center for Type Culture Collection (CCTCC) on Jun. 18, 2023 with the preservation number of CCTCC NO: V202356, the preservation address is Wuhan University, Wuhan, China.

It is another object of the present disclosure to provide a bio-material, which is any one of the following:
A. a nucleic acid molecule represented by the sequence shown as SEQ ID NO:1 or by the complete complement of the SEQ ID NO:1;
B. an expression cassette including the nucleic acid molecule of A;
C. a recombinant vector including the nucleic acid molecule of A;
D. a recombinant microorganism including the nucleic acid molecule of A or the expression cassette of B or the recombinant vector of C;
E. a cell series comprising the nucleic acid molecule of A or the expression cassette of B or the recombinant vector of C.

It is another object of the present disclosure to provide an immunogenic composition including a coxsackievirus group 4 strain as described above or including a biological material as described above.

It is a further object of the present disclosure to provide the use of a coxsackievirus group 4 strain as defined above or a biological material as defined above or an immunogenic composition as defined above, either:
a. the application of the composition in preparing medicines for preventing and/or treating coxsackievirus infection or diseases caused by coxsackievirus infection;
b. the application in preparing a vaccine for preventing and/or treating coxsackievirus infection or diseases caused by coxsackievirus infection;
c. the application in preparing antibodies for preventing and/or treating coxsackievirus infection or diseases caused by coxsackievirus infection;
d. the application in preparing antiserum for preventing and/or treating coxsachievirus infection or diseases caused by coxsachievirus infection;
e. application in preparing a reagent or a kit for detecting coxsachievirus;
f. use in the immunogenicity evaluation of coxsackievirus vaccines for non-diagnostic and therapeutic purposes;
g. use in the protective assessment of coxsackievirus vaccines for non-diagnostic and therapeutic purposes;
h. application in preparing coxsackievirus infected cell model;
i. application in preparing animal model infected by coxsachievirus;
g. the application in the screening of medicines or the evaluation of medicine efficacy for preventing and/or treating coxsackievirus infection or diseases caused by coxsackievirus infection.

It is another object of the present disclosure to provide a vaccine comprising the coxsackievirus B4 strain described above or comprising the biological material described above.

The disclosure also aims to provide a preparation method of the vaccine, which includes the steps of culturing the coxsackievirus B4 strain or the biological material on Vero cells, harvesting the virus solution, inactivating and purifying, and adding an adjuvant to prepare an inactivated vaccine;

Alternatively, attenuated strains can be obtained through adaptive passaging or reverse genetics methods. After cell culturing, the virus solution is harvested, purified, and stabilized with stabilizers and adjuvants to prepare attenuated vaccines.

The beneficial effects are that: the disclosure screens a representative coxsackievirus B4 strain KM140-G01, the strain is separated and cultured by Vero cells, and the cell matrix is safe and reliable as a human vaccine cell matrix. The strain has strong proliferation capability, high genetic stability and good immunogenicity, the genotype D belongs to one of two dominant CVB4 genotypes (D and E) in China, and antisera obtained after animals are immunized by an inactivated vaccine prepared by using the strain as a virulent strain has high neutralizing effect on homologous and heterologous CVB4 isolates of the D and E genotypes. In addition, the disclosure provides a preparation and immunological evaluation method of the inactivated vaccine using KM140-G01 as a virus seed, which lays a solid foundation for developing a safe, effective and economic CVB4 inactivated vaccine. In addition, the KM140-G01 strain can be used for constructing a stable and good-repeatability coxsackievirus B4 intracellular proliferation model and an acute infection animal model, and the models can provide research tools for exploring the pathogenic mechanism of the CVB4 infection, screening antiviral drugs and evaluating vaccine immune protection effects.

### BRIEF DESCRIPTION OF THE DRAWINGS

In order to more clearly illustrate the embodiments of the present disclosure or the technical solutions in the prior art, the drawings that are required to be used in the embodiments or the description of the prior art will be briefly described below, and it is obvious that the drawings in the following description are only embodiments of the present disclosure, and that other drawings can be obtained according to the provided drawings without inventive effort for a person skilled in the art.

FIG. 1 is a schematic diagram showing the adaptation proliferation of CVB4 strain in Vero cells, wherein the left side of FIG. 1 is Vero normal cells, and the right side of FIG. 1 shows the cytopathic effect (CPE) of CVB4 strain;

FIG. 2 shows the plaque morphology of the strain KM140-G01 according to the present disclosure;

FIG. 3 is a CVB4 phylogenetic tree based on the full-length VP1 sequence of the present disclosure; wherein the ⋅ represents the CVB4 isolates of the D and E genotypes involved in the study; the ▴ represents a CVB4 prototype;

FIG. 4 is a graph showing peaks of CVB4 virus concentmicee for liquid chromatography purification according to the present disclosure;

FIG. 5 shows the immunogenicity of an inactivated CVB4 vaccine according to the present disclosure;

FIG. 6 is a model of the prolifemiceion of CVB4 strain on Vero cells according to the present disclosure;

FIG. 7 is a clinical presentation of CVB4 strain infected neonatal BALB/c mice in the present disclosure;

FIG. 8 is a view of HE staining sections of CVB4 strain infected neonatal mice according to the present disclosure;

FIG. 9 is a chart of IHC staining sections of CVB4 strain infected neonatal mice according to the present disclosure;

FIG. 10 shows the viral load of CVB4 strain infected neonatal mice tissue in the present disclosure.

### DETAILED DESCRIPTION OF THE EMBODIMENTS

The following description of the embodiments of the present disclosure will be made clearly and completely with reference to the accompanying drawings, in which it is apparent that the embodiments described are only some embodiments of the present disclosure, but not all embodiments. All other embodiments, which can be made by those skilled in the art based on the embodiments of the disclosure without making any inventive effort, are intended to be within the scope of the disclosure.

### Embodiment 1: Isolation and Characterization of Coxsachievirus Group B4 (CVB4) Strain KM140-G01

### (1) Sample Processing

Collecting feces of children patients with hand-foot-mouth disease in Yunnan Province, China, suspending 1 g to 5 ml with PBS, mixing, centrifuging at 1500 g for 20 min, and filtering and sterilizing the supernatant with 0.22 μm filter membrane.

### (2) Adaptation and Isolation of Viruses on Vero Cells

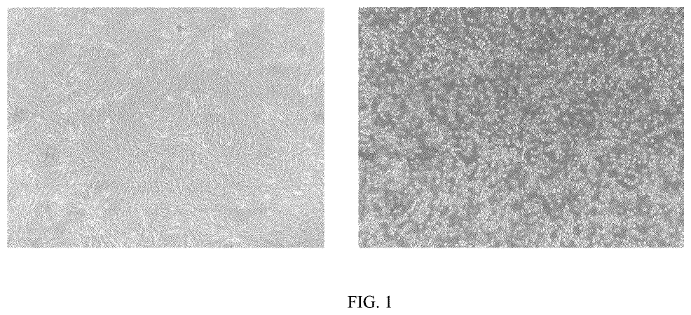

Inoculating 100 μl of filtered solution into monolayer-grown Vero cells, adding MEM culture medium without bovine serum, incubating at 35° C. and 5% co₂ culturing and observing cytopathy, and after the cytopathy appears, harvesting the culture, inoculating Vero cells by the same method for two generations, and freezing the harvested culture for later use, wherein the FIG. 1 is shown.

### (3) Plaque Purification

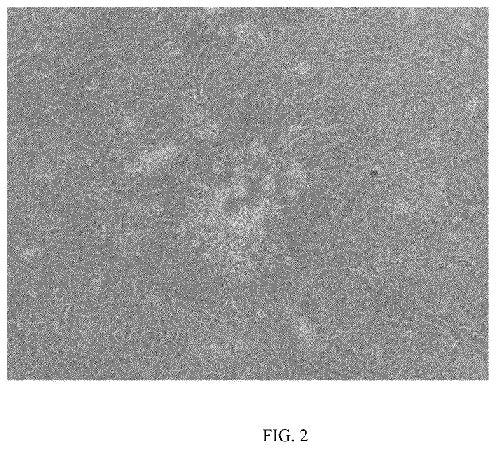

Diluting the virus harvest solution that has been adapted and passaged to the third generation on Vero cells in a 10 fold series to obtain three dilutions with the degree of 10⁻³, 10⁻⁴, 10⁻⁵ and inoculated with monolayer Vero cells, respectively, and adsorbed at 37° C. and 5% co₂ for 1 h, then 1.5% methylcellulose-MEM was added and cultured at 35° C. and 5% co₂ and CPE was observed under a microscope. After the formation of the plaque, the plaque was removed by a pipette and inoculated in monolayer Vero cells and cultured at 37° C. with 5% co₂. and observed CPE under a microscope daily, When CPE reached more than 90%, the virus solution were harvested and continuously inoculated into Vero cells for plaque purification twice by the same method. After three rounds of plaque purification, a monoclonal purified strain was picked up and the single purified wild-type strain of CVB4, was prepared by cultured on Vero cells, see FIG. 2.

### (4) Virus Identification

200 μL of virus solution was extracted and stored in a refrigerator at −80° C. according to the protocol of QIAamp Viral RNA Mini Kit (Qiagen, germany).

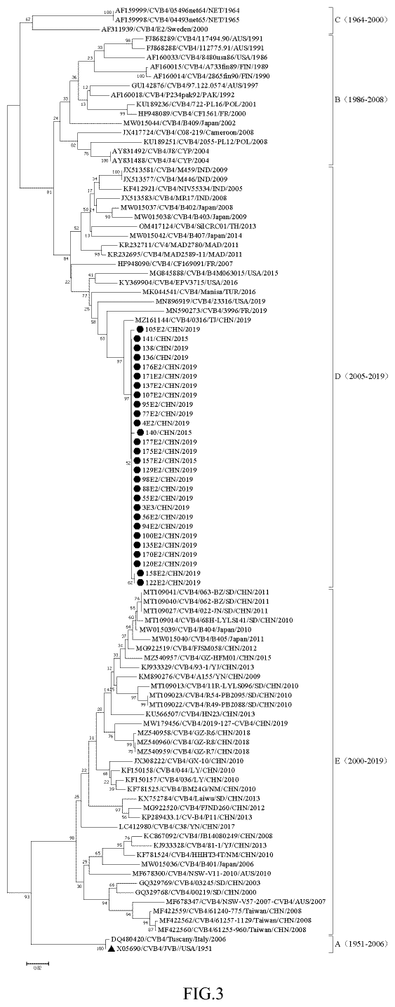

RT-PCR was performed on the above-extracted viral RNA according to the protocol of Primescript™ One Step RT PCR Kit Ver.2 (Takara, Dalian) and using the primer “BOS-BOAS”. The reaction system: the total volume is 20 μL, and each of BOS and BOAS is 1 μL; 2×One Step buffer 10 μL; 3 μl of viral RNA; primeScript 1 Step Enzyme Mix 1 μl; 4 μl of enzyme-free water. RT-PCR reaction conditions: reverse transcription is carried out for 30 min at 50° C.; pre-denaturation at 94° C. for 2 min; denaturation at 94° C. for 30 s, annealing at 52° C. for 30 s, extension at 72° C. for 60 s for 35 cycles; further extension is carried out at 72° C. for 10 min. After 5 μL of the PCR product was identified by electrophoresis on a 1.0% agarose gel, VP1 sequences were obtained by sequencing (Sanger sequencing, ABI373XL sequencer) from Kunming Biotechnology Co., ltd. The virus serotype was determined to be CVB4 by Blast alignment in NCBI. Then, CVB4VP1 sequences obtained in the GenBank database were aligned using MEGA7.0 software, primers B4VP1F and B4VP1R were designed according to the conserved regions, and Primer-BLAST in the NCBI database was used to detect Primer specificity. The CVB4 full length VP1 sequence was sequenced using B4VP1F and B4VP1R. A phylogenetic tree was constructed using the maximum likelihood method (Maximum Likelihood, ML) of MEGA7.0 and Kimura 2-parameter model, and the number of Bootstrap repeats was set to 1000 for evaluating the reliability and repeatability of the phylogenetic tree, and the phylogenetic analysis was performed on the CVB4 strain isolated this time. The strain KM140-G01 belongs to the genotype D, and is one of the main genotypes CVB4 in China, and is shown in FIG. 3.

The whole genome sequence sequencing primers and amplification positions involved are as follows (wherein Y represents C or T, N represents A or C or G or T, R represents A or G):

B-OS (upstream primer): 5′-GGYTAYATNCANTGYTGGTAYCARAC-340 , SEQ ID NO: 2, amplification 2296-2321;

B-OAS (downstream primer): 5′-GGTGCTCACTAGGAGGTCYCTRTTRTARTCYTCCCR-3′, SEQ ID NO:3, amplifications 3414-3379;

EVIF (upstream primer): 5′-TTAAAACAGCCTGTGGGTTG-3′, SEQ ID NO:4, amplifying 1-20;

B41f (upstream primer): 5′-GTACCAGAAGCCGAAATGG-3′, SEQ ID NO:5, amplifying 863-882;

B41r (downstream primer): 5′-CTAGCACTCAGACTAGTC-3′, SEQ ID NO:6, amplifying 774-752;

B42f (upstream primer): 5′-GATACGTGGTGGATGAT-3′, SEQ ID NO:7, amplifying 774-752;

B42r (downstream primer): 5′-GGCCTCTAGCAATTGTGTC-3′, SEQ ID NO:8, amplifying 2356-2338;

B43f (upstream primer): 5′-TATATGGTTACAACTCACT-3′, SEQ ID NO:9, amplifying 2912-2930;

B44f (upstream primer): 5′-TGTGGCTAGAAGATGATG-3′, SEQ ID NO:10, amplification 3557-3574;

B45f (upstream primer): 5′-AGCTCAACAGCTCGGTGT-3′, SEQ ID NO:11, amplification 4328-4345;

B45r (downstream primer): 5′-GGAGCTTTCAATGAACTC-3′, SEQ ID NO:12, amplifying 5778-5761;

B46f (upstream primer): 5′-ACCTGAATCCAGGCATAG-3′, SEQ ID NO:13, amplification 6347-6364;

B46r (downstream primer): 5′-TTGTGGGAGTATCCTAGC-3′, SEQ ID NO:14, amplification 6533-6516;

B47f (upstream primer): 5′-GAATCAATCAGGTGGACC-3′, SEQ ID NO:15, amplifying 7105-7122;

B4VP1F (upstream primer): 5′-TAATGTGTCTTGTGTCGGCT-3′, SEQ ID NO:16, amplification 2207-2226;

B4VP1R (downstream primer): 5′-TTGCCAATCAAAACTTGTTG-3′, SEQ ID NO: 17, amplification 3222-3203;

EV8R (downstream primer): 5′-CACCGAATGCGGAGAATTTA-3′, SEQ ID NO:18.

Amplifying all fragments of the whole genome of the virus by utilizing the primers, sequenceing and splicing to obtain the nucleotide sequence of the whole genome. The nucleotide sequence of the whole genome of CVB4 isolate KM140-G01 obtained in the embodiment is shown in SEQ ID NO: 1.

CVB4 strain KM140-G01 is preserved in China center for type culture Collection on Jun. 16, 2023, with a preservation number of CCTCC NO: V202356, the preservation address is Wuhan University, Wuhan, China.

### (5) Virus Infectivity Titer Detection

The infectious titers of the CVB4 strain KM140-G01 were determined using a microtissue culture technique. CVB4 Virus was serially diluted 10 folds with virus dilution (serum-free MEM Medium) to obtain virus fluid of 10⁻¹˜10⁻⁸ and 100 μL of the virus solution at different dilutions per well were added into a 96 well plate. At least 8 wells were left as controls. 100 μL of Vero cell suspension (10⁴ cells/well) were inoculated in 96-well plates and incubated at 35° C. and 5% co₂ for 7 days and observed under a microscope. And 50% cell culture infectious dose (CCID₅₀) was calculated by Karber method. The calculation formula is as follows: 1gCCID₅₀=L+d (S−0.5), L is the logarithm of the lowest dilution factor of the virus, d is the dilution factor, S is the sum of cytopathic aperture ratios.

Results: the titres of the strain KM140-G01 virus are as 7.251 g CCID₅₀/ml.

### (6) Analysis of Genetic Stability

The culture supernatant was harvested from P1 to P15 passages by multiple subcultures on Vero cells. The viral RNA was extracted from the P1, P5, P10 and P15 generation virus harvests according to the protocol of QIAamp Viral RNA Mini Kit (Qiagen, Germany). The whole viral genome was amplified by using the primers described in step (4) according to the protocol of Primescript™ One Step RT PCR Kit ver.2 (Takara, Dalian). RT-PCR reaction conditions: reverse transcription at 50° C. for 30 min; pre-denaturation at 94° C. for 2 min; denaturation at 94° C. for 30 s, renaturation at 52° C. for 30 s, extension at 72° C. for 50 s within a total 35 cycles; further extension is carried out at 72° C. for 5 min. The PCR product was sent to Kunming Qingke biotechnology Co.Ltd for sequencing (Sanger sequencing, ABI373XL sequencer) and the sequencing primers were set forth in step (4). The whole genome sequences were aligned and spliced using SeqMan of DNAStar7.1 software to obtain the whole genome sequences of each generation. Nucleotide and amino acid changes of the whole genome sequence for different generations were analyzed by using MEGA7.0 software.

The KM140-G01 strain showed 5 nucleotide mutations during passage, and the mutations are respectively located at 447, 1166, 4251, 4342 and 4390 of the CDS region in the sequence. Two amino acid mutations appeared in the P5 generation with the 389th and 1448th amino acids in the CDS region being mutated to K→R and S→A respectively, and are located in VP3 and 3A sections. Three amino acids mutations appeared in the P15 generation with the 149th, 1417th and 1464th in the CDS region being mutated into W→G, S→R and Y→H respectively, and are located in VP2, 2C and 3A sections respectively, see table 1. The genetic stability results show that the KM140-G01 strain has fewer mutation sites and has no mutation in VP1 segment in the continuous passage process, and the better genetic stability is shown.

TABLE 1
The nucleotide and amino acid sequence changes of the whole
genome sequences of different generations of KM140-G01 strain

| | | | | Nucleotides or amino acids | | | |
|---|---|---|---|---|---|---|---|
| | Location | Amino | | of different generations | | | |
| Strain | Nucleotide | acid | Section | P1 | P5 | P10 | P15 |
| KM140-G01 | 447 | / | VP2 | G | G | G | A |
| | 1166 | / | VP2 | A | G | G | G |
| | 4251 | / | 2C | T | T | T | A |
| | 4342 | / | 3A | T | G | G | G |
| | 4390 | / | 3A | T | T | T | C |
| | / | 149 | VP2 | W | W | W | R |
| | / | 389 | VP3 | K | R | R | R |
| | / | 1417 | 2C | S | S | S | R |
| | / | 1448 | 3A | S | A | A | A |
| | / | 1464 | 3A | Y | Y | Y | H |

### Embodiment 2: Preparation of CVB4 Inactivated Vaccine by Using KM140-G01 Strain as Virus Seed

### 1. Preparation of CVB4 Inactivated Vaccine

### (1) Virus Culturing

Vero cells were cultured to the desired quantity using 10-layer cell factories or T225 cell culture flasks under the following conditions: MEM medium containing 10% new born bovine serum, standing and culturing at 37° C., and 5% co₂. Then Vero cells (90%-95% confluent monolayer) were infected by coxsachievirus B4 strain KM140-G01 at a multiplication of infection (MOI) of 0.01-0.1, and cultured in MEM medium without bovine serum at 35° C. and, 5% co₂ for 2-4 days until CPE reaches over 90%, and harvesting virus solution, namely the virus harvest solution.

### (2) Virus Inactivation

performing repeated freeze-thaw cycles at room temp and −20° C. for 3 times, and inactivating the virus harvest solution by formaldehyde with the final concentration of 100 μg/ml at 37° C. to obtain the inactivated virus solution. Taking a virus solution sample for inactivating 144 hours, neutralizing by sodium bisulphite. The final inactivated sample was inoculated with Vero cells and cultured continuously for 3 times to verify the inactivation effect. No CPE was observed, indicating that the above concentration of formaldehyde completely inactivated the virus within 144 h (6 days). To better ensure the thoroughness of virus inactivation, the inactivation period was set to 7 days.

### (3) Clarifying, Concentrating and Purifying Inactivated Virus Solution

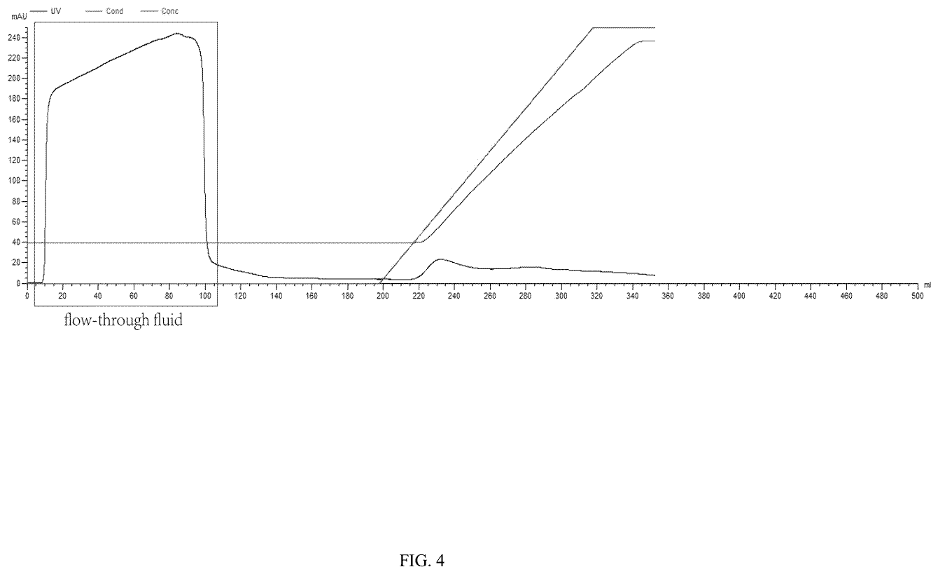

Centrifuging the inactivated virus solution at 8000 rpm for 30 min, and removing cell debris; performing tangential flow filtration on the clarified virus solution by adopting an ultrafiltration membrane bag with the aperture of 100 kD, and concentrating for 50-200 times to prepare virus concentrated solution; purifying with Capto Core 400 chromatographic column, collecting the flow-through liquid, which is the virus purified liquid, as shown in FIG. 4. The virus purified solution is filtered and sterilized by a 0.22 um filter to obtain the original virus solution.

### (4) Preparation of Inactivated Vaccine

Adding original virus solution, aluminum adjuvant and diluent (PBS) with corresponding volumes according to the designed volumes, antigen doses and adjuvant concentrations, and uniformly mixing to obtain the CVB4 inactivated vaccine. In the test for evaluating the immunogenicity of the vaccine, the vaccine volume was 0.1 ml/dose, the antigen content was 3-20 ug/0.1 ml, and the aluminum content concentration was 0.35 mg/ml.

### 2. CVB4 Virus Neutralizing Antibody Detection

Neutralization experimental method: after the virus infects permissive cells, it causes morphological changes and CPE. When a specific neutralizing antibody binds to the virus, it loses infectivity, and CPE is inhibited.

### (1) Attack Virus CCID₅₀ Titration and Titer Gradient Preparation

The virus was titrated 2-3 times according to the procedure of step 1 above, and the average value was taken to determine 100CCID₅₀ per 0.05 ml, the attack virus was diluted in the calculated dilution ratio to obtain the total amount of virus required for the experiment (100 CCID₅₀ per 0.05 ml). Taking 3 small tubes, adding 0.9 ml of virus diluent into each tube, and sucking 0.1 ml of diluted attack virus solution into the first small tube (i.e. 10CCID₅₀ per 0.05 ml) and replacing the tip, mixing uniformly, and diluting to 1CCID₆₀/0.05 ml and 0.1CCID₅₀/0.05 ml in sequence according to the method, and a virus titer comfirmation test was performed.

### (2) Diluting Serum to be Tested

The serum to be detected is frozen at −20° C. for preparation and detection. Placing a plurality of sterile small tubes on a test tube rack, adding 0.3 ml of diluent into each tube, adding 0.1 ml of serum to be detected, covering a cover, shaking and mixing uniformly to obtain the diluted serum with the concentration of 1:4. Inactivating at 56° C. for 30 min. And opening the sterile packaging 96-well tissue culture plate, adding 0.05 ml of diluent into each well, carrying out 2-time serial dilution on each serum sample, making two compound wells for each dilution of each serum sample, and simultaneously setting two wells of each serum control well and cell control well to be tested.

### (3) The Virus Neutralizing Antibody Assay Procedure:

0.05 ml attack virus (containing 100CCID₅₀/0.05 mL) was added to each of the diluted serum 96-well plates and incubated at 37° C. for 2 h. A new 96-well plate was used for virus drip-back assay (which must be done for each experiment). After the incubation, the cells were digested with pancreatin to prepare a cell suspension, and the concentration of the cell suspension was adjusted to 2×10⁵ and 0.1 mL of cell suspension is respectively added into each well of the sample plate to be detected and the virus titer comfirmation assay plate, and the mixture is placed at 35° C. and 5% co₂. The final results were determined 7 days in culture and virus titration results were recorded. Note that: if the virus drop-back result is not in the range of 32-320CCID₅₀, the test is not effective and a retry is required.

### (4) Result Determination

When CPE appears in 1 well of the 2 wells of the serum with the highest dilution while CPE does not appear in the other wells, the reciprocal of the dilution is the neutralizing antibody titer of the serum sample; when CPE appears in the high dilution 2 wells and CPE does not appear in the adjacent low dilution 2 wells, the reciprocal of the average dilution of the two wells is the neutralizing antibody titer of the serum sample; when CPE appears in 1 well of two adjacent dilutions of serum and CPE does not appear in the other 1 well, the reciprocal of the average dilutions of the two wells is the neutralizing antibody titer of the serum sample.

### 3. Immunogenicity Evaluation of CVB4 Inactivated Vaccine

(1) The vaccine antigen prepared according to the method of the step 1 was immunized on BALB/c mice, and the immunization effect of different doses of antigen was analyzed, wherein 0.1 mL of vaccine antigen contained 3 μg, 5 μg, 10 μg, 20 μg. Immunization procedure: 3-needle subcutaneous injection immunization, 0.1 ml/mouse, blood collection after 1, 2 and 3 serum separation, and detection of neutralizing antibodies. During the experiment, BALB/c mice were divided into 6 groups of 10 mice each, and the controls were an aluminum adjuvant group and PBS buffer group, respectively.

The neutralizing antibody assay protocol is as follows:
serum was diluted 1:8, 1:16, 1:32, 1:64, 1:128, 1:256, 1:1024, 1:2048, 1:4096; positive control: rabbit polyclonal antisera prepared in this laboratory; negative control: rabbit negative serum prepared in this laboratory. Serum neutralizing antibody titers after immunization of each experimental group and control group were detected according to the neutralizing antibody detection method in step 2.

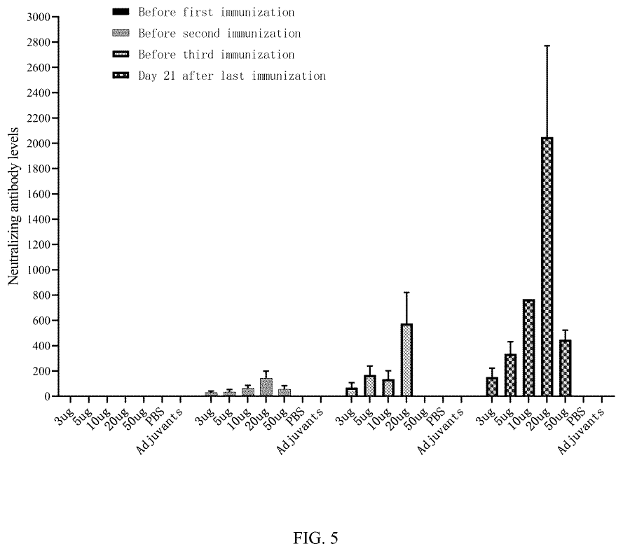

The results show that: the dose-effect relationship of the vaccine is remarkable, the neutralization titer is increased along with the increase of the dose, the neutralization antibody titer of KM140-G01 after 3-phase immunization is more than 1:152, and the protective antibody level is achieved, as shown in FIG. 5.

(2) The immune effect of the vaccine prepared by KM140-G01 on other isolates of the same genus D genotype and CVB4 strains of the E genotype was tested using the other 3D genotype CVB4 isolates isolated in the laboratory, 1E genotype CVB4 and serum after 20 ug dose group 3.

The results show that: After the third immunisations, the neutralization titers of antiserum induced in the 20 μg groups against other D and E genotype CVB4 strains were more than 1:1024, and the protective effect exists.

### 4. Evaluation of Protective Efficacy of CVB4 Inactivated Vaccine

To study the immunoprotection of maternal antibodies against neonatal mice for challenge infection, a CVB4 vaccine of 20 μg/100 μL of the experimental group, and PBS and aluminum adjuvant of the control group were set. 8 week old BALB/c mice were immunized with 5 animals per group. After the first immunization of the female mice, the female mice are allowed to be caged with the male mice, and after the pregnancy of the female mice, the female mice are immunized for the second time, and about two weeks after the second immunization. Three days after delivery, the 3-day-old mice were subjected to a challenge experiment, and the brain of each mice was injected with a lethal dose (30 μL, virus content 10^{5.5}CCID₅₀). The protective effect of maternal antibodies was assessed by daily observation of body weight changes and clinical scores of each group of suckling mice with respect to the 4 D genotype CVB4 isolate virus solution involved in step 3.

The results show that the body weight of the suckling mice inoculated by the experimental female mice group is normally increased after virus inoculation, the suckling mice have no clinical symptoms and are healthy, and the final survival rate is 100%. The weight of the control inoculated suckling mice is hardly increased after virus inoculation, clinical symptoms are obvious, the mice begin to die on the 2nd day, all the mice die on the 3 rd day, and the final survival rate is 0%. The above data indicate that the maternal antibodies of the experimental group can provide the suckling mice with complete protection against lethal doses of CVB4.

### Embodiment 3: Preparation of Infected Cell Model by CVB4 Virus Strain KM140-G01

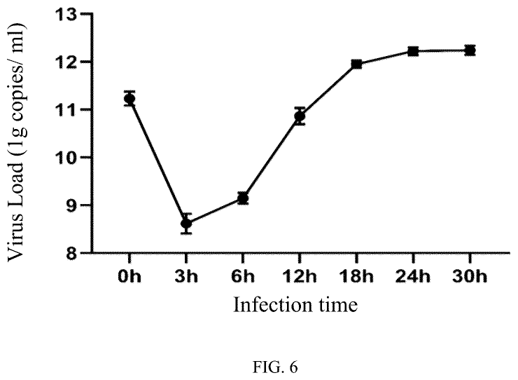

Vero cells were cultured to a monolayer, the cell culture medium was removed, washed with PBS, and KM140-G01 virus was inoculated at MOI=1, at 37° C. and 5% co₂. Adsorbing for 1 hr, removing virus containing solution, washing cell surface with PBS for 3 times, adding MEM culture medium without bovine serum, and incubating at 5% co₂ and, 35° C. Samples were taken at 0 hr, 3 hrs, 6 hrs, 12 hrs, 18 hrs, 24 hrs and 30 hrs post infection, with the addition of virus solution as 0 hr time point of post infection, and RNA samples were extracted. Using primers CVB4VP1-F3 (TGT CCT GTT TCC ACT GCT GTT, SEQ ID NO: 19), CVB4VP1-R3 (GAA GAA TCA GTG GAG CGT GC, SEQ ID NO: 20) and Taqman probes CVB4VP1-P3 (CTC TGA ACA AAT CC 5′FAM 3′MGB,SEQ ID NO:21) designed based on the conserved sequences of CVB4 virus VP1 and CVB4 RNA standards prepared in the present laboratory, virus copy numbers were detected using RT-qPCR to determine the duration of a complete infection cycle of KM140-G01 on Vero and to establish a one-step growth curve on Vero cells. The proliferation model of strain KM140-G01 on Vero cells is shown in FIG. 6.

RT-qPCR reaction System (20 μL):
2×one-step RT-PCR Buffer III Buffer 10 μL, TaKaRa Ex Taq HS (5U/μL) 0.4 μL, primeScript RT Enzyme Mix II 0.4 μL, CVB4vp1-P3 (10 μmol/L) 0.4 μL, CVB4vp1-F3 (10 μmol/L) 1 μL, CVB4vp1-R3 (10 μmol/L) 1 μL, non-enzymatic water 5.8 μL, RNA template 1 μL.

Reaction conditions: reverse transcription is carried out for 5 min at 42° C.; pre-denaturation at 95° C. for 10 s, denaturation at 95° C. for 5 s, annealing at 60° C. for 30 s (40 cycles).

### Embodiment 4: Preparation of Infection Animal Model by CVB4 Virus Strain KM140-G01

By Intracerebral injection (IC) route, respectively infect 3-day-old BALB/c neonatal mice with three different infection doses of KM140-G01 virus of 10^{3.5}, 10^{4.5}, 10^{5.5}CCID₅₀, and the best infection dose for establishing a CVB4 infection model of the 3-day-old BALB/c neonatal mice was explored.

| Clinical scoring | clinical manifestation |
|---|---|
| 0 points | health |
| 1 points | somnolence, listlessness |
| 2 points | emaciation |
| 3 points | limb weakness, hair rarefaction, |
| | humpback or weakness |
| 4 points | dying or death |

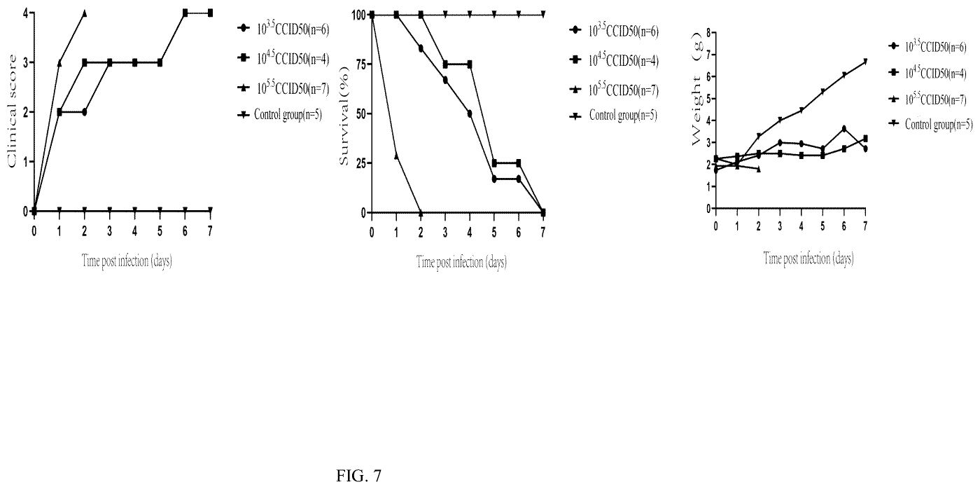

The body weight, survival rate, and clinical score of the mice of the same age in the control group are not significantly different. Experiment group 10^{5.5}CCID₅₀. The mice in the experimental group with the dose of 10^{5.5}CCID₅₀ experienced weight loss after the attack of the virus. On the first day, the symptoms of the disease were severe, and on the second day, the mice in the experimental group were on the brink of death or dying. The survival time of the mice in the experimental group was 2-3 days. The body weight of the experimental group with the dose of 10^{3.5} and 10^{4.5}CCID₅₀ was not increased basically, and the growth state is poorer compared with that of the control group. The experimental group of the suckling mice showed symptoms of illness after 1-2 days, and the survival time of the suckling mice did not exceed 7 days, see FIG. 7.

Experimental mice infected with the KM140-G01 virus at three different infection doses of 10^{3.5}, 10^{4.5}, 10^{5.5}CCID₅₀ developed into a moribund state, active virus were obtained from their brains, hearts, lungs, spleens, pancreas, upper limb muscles, and intestinal tracts. After the remaining organs were fixed in 10% neutral formalin for 48 hours, paraffin sections were prepared by paraffin embedding for HE staining and Immunohistochemical (IHC) detection. For immunohistochemical detection, paraffin sections are baked for 1 h at 60° C., subjected to alcohol gradient dewaxing, EDTA antigen repair and dehydration, incubated overnight at 4° C. by using the CVB4 polyclonal antibody prepared in the laboratory, incubated at room temperature for 30 min, and then DAB developed and hematoxylin anti-blue are added dropwise. Negative control group section Negative serum was used for the antibody. The results were examined under a microscope.

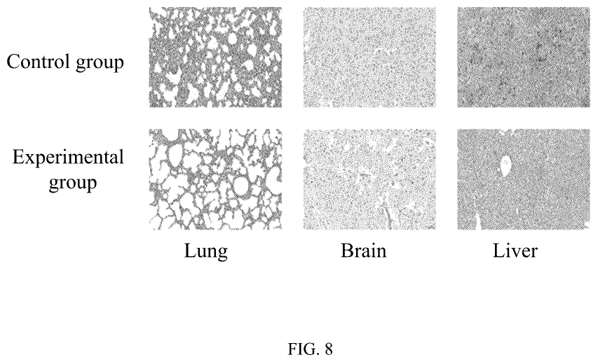

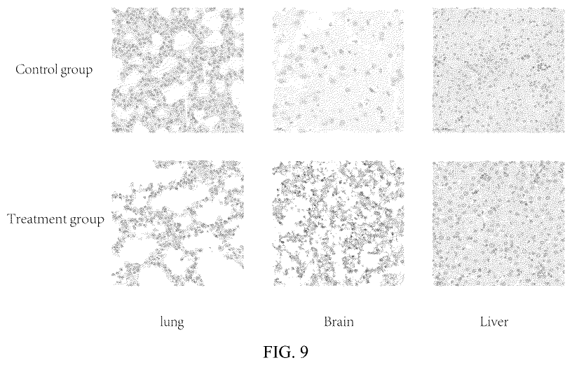

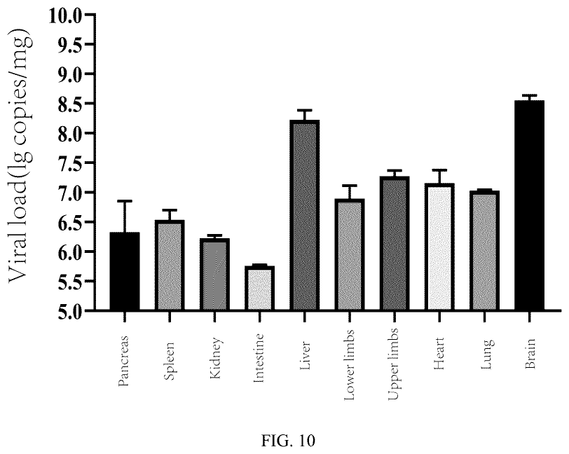

HE staining showed severe pathological changes in lung, brain and liver in the 3 dose experimental group, specific pathological changes were manifested as: the lung has more alveoli with slight expansion, interfusion and volume increase, and with larger area bleeding, a large amount of red blood cells are visible in the alveoli space. The brain has cortex and hippocampal region structural disorder, and multiple neurons are necrotized; more red blood cells are visible from hemorrhage of cortex, hippocampus and meninges; thalamus rarely necroses, and nuclei shrink deeply, disintegrate or dissolve. The liver tissue is necrotic at multiple places, has a disordered structure, is mostly necrotic cell fragments, and is accompanied by small amount of granulocyte infiltration around; a plurality of extramedullary hematopoietic cells are distributed in a range shape of 10^{5.5}CCID₅₀. The dose group is shown in FIG. 8. The results of immunohistochemical experiments showed that the maximum dose (10^{5.5}CCID₅₀) challenge, CVB4 antigen distribution appeared in the lung, brain and liver of the suckling mice, and other tissues did not see obvious CVB4 antigen distribution, see FIG. 9. Collecting 10^{5.5}CCID₅₀ and the viral load of each tissue of the mice was measured by using a one-step real-time fluorescent quantitative RT-PCR reaction according to embodiment 3, and the measurement results show that the viral load level of brain and liver in the experimental group is significantly higher than that of other tissues, as shown in FIG. 10.

In the present specification, each embodiment is described in a progressive manner, and each embodiment is mainly described in a different point from other embodiments, and identical and similar parts between the embodiments are all enough to refer to each other.

The previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

What is claimed is:

1. A coxsackievirus group B4 strain, wherein the strain has a genomic sequence as set forth in SEQ ID NO: 1 or a complete complement of the SEQ ID NO:1.

2. The coxsackievirus B4 strain of claim 1, wherein the strain is named as KM140-G01, preserved at Chinese Collection for Culture Collection on Jun. 18, 2023, with a preservation number of CCTCC NO: V202356, the preservation address is Wuhan University, Wuhan, China.

3. A biomaterial characterized by being any one of the following:
A. a nucleic acid molecule with the sequence shown in the SEQ ID NO:1 or the complete complement of the SEQ ID NO:1;
B. an expression cassette comprising the nucleic acid molecule of A;
C. a recombinant vector comprising the nucleic acid molecule of A;
D. a recombinant microorganism comprising the nucleic acid molecule of A or the expression cassette of B or the recombinant vector of C;
E. cell series comprising the nucleic acid molecule of A or the expression cassette of B or the recombinant vector of C.

4. An immunogenic composition comprising a coxsackievirus group B4 strain of claim 1.

5. An immunogenic composition comprising a coxsackievirus B4 strain of claim 2.

6. An immunogenic composition comprising the biomaterial of claim 3.

7. Application of a coxsackievirus group B4 strain of claim 1, characterized in that it is any one of the following:
a. application in preparing medicine for preventing coxsachievirus infection or diseases caused by coxsachievirus infection;
b. the application of the compound in preparing a vaccine for preventing coxsackievirus infection or diseases caused by coxsackievirus infection;
c. the application of the antibody in preparing the antibody for preventing the coxsachievirus infection or the diseases caused by the coxsachievirus infection;
d. the application of the anti-serum for preventing coxsachievirus infection or diseases caused by coxsachievirus infection;
e. application in preparing a reagent or a kit for detecting coxsachievirus;
f. use in an evaluation of immunogenicity of a coxsackievirus vaccine for non-diagnostic and therapeutic purposes;
g. use in the protective assessment of coxsackievirus vaccines for non-diagnostic and therapeutic purposes;
h. application in preparing cell model infected by coxsackievirus;
i. application in preparing animal model infected by coxsachievirus; or
j. application in medicine screening for preventing coxsachievirus infection or diseases caused by coxsachievirus infection;
wherein the coxsachievirus is group B4 strain, D genotype and/or group B4 strain, E genotype.

8. Application of a coxsackievirus group B4 strain of claim 2, characterized in that it is any one of the following:
a. application in preparing medicine for preventing coxsachievirus infection or diseases caused by coxsachievirus infection;
b. the application of the compound in preparing a vaccine for preventing coxsackievirus infection or diseases caused by coxsackievirus infection;
c. the application of the antibody in preparing the antibody for preventing the coxsachievirus infection or the diseases caused by the coxsachievirus infection;
d. the application of the anti-serum for preventing coxsachievirus infection or diseases caused by coxsachievirus infection;
e. application in preparing a reagent or a kit for detecting coxsachievirus;
f. use in an evaluation of immunogenicity of a coxsackievirus vaccine for non-diagnostic and therapeutic purposes;
g. use in the protective assessment of coxsackievirus vaccines for non-diagnostic and therapeutic purposes;
h. application in preparing coxsackievirus infected cell model;
i. application in preparing animal model infected by coxsachievirus; or
j. application in medicine screening for preventing coxsachievirus infection or diseases caused by coxsachievirus infection;
wherein the coxsachievirus is group B4 strain, D genotype and/or group B4 strain, E genotype.

9. Application of the biomaterial of claim 3, characterized in that it is any one of the following:
a. application in preparing medicine for preventing coxsachievirus infection or diseases caused by coxsachievirus infection;
b. the application of the compound in preparing a vaccine for preventing coxsackievirus infection or diseases caused by coxsackievirus infection;
c. the application of the antibody in preparing the antibody for preventing the coxsachievirus infection or the diseases caused by the coxsachievirus infection;
d. the application of the anti-serum for preventing coxsachievirus infection or diseases caused by coxsachievirus infection;
e. application in preparing a reagent or a kit for detecting coxsachievirus;
f. use in an evaluation of immunogenicity of a coxsackievirus vaccine for non-diagnostic and therapeutic purposes;
g. use in the protective assessment of coxsackievirus vaccines for non-diagnostic and therapeutic purposes;
h. application in preparing coxsackievirus infected cell model;
i. application in preparing animal model infected by coxsachievirus; or
j. application in medicine screening for preventing coxsachievirus infection or diseases caused by coxsachievirus infection;
wherein the coxsachievirus is group B4 strain, D genotype and/or group B4 strain, E genotype.

10. Application of the immunogenic composition of claim 4, characterized in that it is any one of the following:
a. application in preparing medicine for preventing coxsachievirus infection or diseases caused by coxsachievirus infection;
b. the application of the compound in preparing a vaccine for preventing coxsackievirus infection or diseases caused by coxsackievirus infection;
c. the application of the antibody in preparing the antibody for preventing the coxsachievirus infection or the diseases caused by the coxsachievirus infection;
d. the application of the anti-serum for preventing coxsachievirus infection or diseases caused by coxsachievirus infection;
e. application in preparing a reagent or a kit for detecting coxsachievirus;
f. use in an evaluation of immunogenicity of a coxsackievirus vaccine for non-diagnostic and therapeutic purposes;
g. use in the protective assessment of coxsackievirus vaccines for non-diagnostic and therapeutic purposes;
h. application in preparing coxsackievirus infected cell model;
i. application in preparing animal model infected by coxsachievirus; or
j. application in medicine screening for preventing coxsachievirus infection or diseases caused by coxsachievirus infection;
wherein the coxsachievirus is group B4 strain, D genotype and/or group B4 strain, E genotype.

11. A vaccine comprising an inactivated vaccine or attenuated vaccine prepared from the coxsackievirus group B4 strain of claim 1.

12. A vaccine comprising an inactivated vaccine or attenuated vaccine prepared from the coxsackievirus group B4 strain of claim 2.

13. A vaccine comprising an inactivated vaccine or attenuated vaccine prepared from the biomaterial of claim 3.

14. A method for preparing the vaccine, wherein the coxsackievirus B4 strain is cultured on Vero cells, the virus solution is harvested, and after inactivation and purification, the inactivated vaccine is prepared by adding an adjuvant; or obtaining the attenuated strain with attenuated toxicity by adaptive passage or reverse genetics method, culturing cells, collecting virus solution, purifying, and adding stabilizer and adjuvant, wherein the vaccine prepared by the preparation method is the vaccine of claim 11

15. A method for preparing the vaccine, wherein the coxsackievirus B4 strain is cultured on Vero cells, the virus solution is harvested, and after inactivation and purification, the inactivated vaccine is prepared by adding an adjuvant; wherein the coxsackievirus B4 strain is named as KM140-G01, preserved at Chinese Collection for Culture Collection on Jun. 18, 2023, with a preservation number of CCTCC NO: V202356, the preservation address is Wuhan University, Wuhan, China or obtaining the attenuated strain with attenuated toxicity by adaptive passage or reverse genetics method, culturing cells, collecting virus solution, purifying, and adding stabilizer and adjuvan,.wherein the vaccine prepared by the preparation method is the vaccine of claim 11.

16. A method for preparing the vaccine, wherein the biomaterial is cultured on Vero cells, the virus solution is harvested, and after inactivation and purification, the inactivated vaccine is prepared by adding an adjuvant; or obtaining the attenuated strain with attenuated toxicity by adaptive passage or reverse genetics method, culturing cells, collecting virus solution, purifying, and adding stabilizer and adjuvant, wherein the vaccine prepared by the preparation method is the vaccine of claim 11


SEQUENCE LISTING (21 sequences)

SEQ ID NO: 1 - DNA, 7461 bp, unidentified
  source 1..7461: mol_type = other DNA; note = coxsackievirus
gggttgttcccacccacagaggccacagggcgctagcacactggtatcccggtacctttgtgcgcctgttttatccacccctcccccaaaagaaacttagaagtttgactacaacggtcaataggaagcccagcaaaccaactgggtcatgaccaagcacttctgtaaccccggactgagtatcaataagctgctcacgcggctgaaggagaaaacgttcgttacccggccaattacttcgagaaacctagtaccaccatgaaagttgcgcggcgtttcgctccgcacaaccccagtgtagatcaggtcgatgagtcaccgcgttcctcacgggtgaccgtggcggtggctgcgttggcggcctgcccatggggcaacccatgggacgcttcaatactgacatggtgcgaagagtctattgagctaattggtagtcctccggcccctgaatgcggctaatcctaactgcggagcagatgcccacatgccagtgggttgtctgtcgtaacgggtaactctgcagcggaaccgactactttgggtgtccgtgtttccttttattcttatactggctgcttatggtgacaattgagagattgttaccatatagctattggattggccatccggtatctaacagagcaatcatttatctgtttgttgggttcgtacccttgaattataaagtcttcaacactcttaattttgtattacaacaaaactcagcaaaatgggagctcaggtctcaacccaaaagacaggggcacacgagactagtctgagtgctagtggtaactcagttatccattacaccaacataaattactataaggatgctgcatcgaactcggcaaacaggcaggattttacccaggaccctagcaagttcacagaaccggtcaaggatgttatgataaaatcactaccagcactcaactcaccaactgtggaggaatgtgggtatagtgacagggttagatcaataacactaggaaactcaaccataacaacacaggagtgtgctaacgttgtggtgggatatggtgtgtggcctgactatcttagtgatgaagaagcgacagcagaggatcaacctacccaaccagatgtcgcaacgtgcaggttctatacattgaactcagtgaaatgggagatgcaatcagcagggtggtggtggaagttcccagacgcattatcagagatgggtcttttcggacaaaatatgcagtaccactatcttggcagatcaggatacacaattcacgtacagtgcaatgcttctaaatttcatcaggggtgcctactggttgtgtgtgtaccagaagccgaaatgggatgcactaatgttgaaaatgcgccctcgtacggcgacctatgcggtggggagacagcaaagcagtttgaacaaaatgccaccacaggtgaaactgccgttcagacggcagtgtgcaacgcgggaatgggtgtgggggttggcaacctgaccatatacccgcaccagtggatcaatttgaggaccaacaacagtgccaccattgtgatgccctacattaacagcgtcccgatggataatatgtttagacacaacaattttacattgatgataataccatttgcaccattggattatgttactggagcatcctcatacatccccatcaccgttacagtagccccaatgaatgctgaatacaacgggttgcggctggccggtcatcaaggactaccgactatgctcacaccaggtagcacacaatttctgacctcagatgacttccagtccccctcagcaatgccacagttcgacgtgactcctgagatgaacatcccagggcaagtcaggaacttgatggagatagcagaagttgattctgtggtgccacttaataatctgaaagctaatttgaaaaccatggaagcttacagggttcaagtaagatcaatggatgaaatgggtggccaaatatttgggttccctctacaaccaggagcatcaagcgtgctgcagcggacattgttgggagaaatattaaattactatacacattggtcagggagcctgaaactgacgtttgtgttctgtggttcagccatggctactggaaagttcttgcttgcctattcaccacctggcgcaggagccccagatagcaggaaaaatgcaatgctggggacacacgtcatatgggatgttggattacaatccagctgtgtgctctgtgtaccatggattagccaaacacactacagatacgtggtggatgataaatacacagccagtgggttcatatcatgttggtaccagaccaatgtcatcgtcccagcagaagcacagaaatcctgctacataatgtgtcttgtgtcggcttgcaacgacttttcagttcgcatgttaagggatacgcaatttattaagcagaacaacttttaccaagggccaacagaagaatcagtggagcgtgcaatgggcagggtcgcagacacaattgctagaggcccgtcgaactctgaacaaatccccgctctaacagcagtggaaacaggacacacatcacaggttgatccaagtgacaccatgcaaaccaggcacgtgcacaattatcactcaaggtctgaatcatcaattgagaatttcttgtgcagatctgcttgtgtaatttacatcaaatattcaagcgctgagtccaacaacttaaaacgttatgcggagtgggtcattaacacaagacaggtggcgcagctgcgacggaaaatggaaatgttcacatacattcgctgtgacatggagttgacatttgtcataaccagccaccaggaaatgtccacagctaccaactctgatgtcccggttcaaacacaccaaataatgtacgtgccacctggaggccccgtgccaacatcagtgaatgattacgtgtggcaaacatcaaccaaccctagtattttttggacagagggaaatgcaccaccaagaatgtctataccatttatgagtataggcaatgcatacacgatgttctatgacggttggtcaaatttctccagggatggcatatatggttacaactcactaaataacatggggaccatctatgcgcgccacgttaatgattccagtcctggtggcttgaccagcaccatccgcatatacttcaagccaaaacacgtcaaggcgtacgtgccgcgtccaccacgcttgtgccaatacaagaaggcaaaaaatgtaaattttgatgttgaagccatcaccactaaacgtgcaagtttggttaccactggcccatgtgggcagcagtctggagctgtgtatgtaggcaactacagaattataaacagacacttagcaacaagttttgattggcaaaattgcgtgtgggaagactataacagagaccttctagttagcacaactaccgctcatgggtgtgatgttattgcaaggtgccagtgcacatccggcgtttacttctgtacatccagaaacaaacactatcccgtgatttttgagggcccaggtttggtggaagtccaagagagtgaatattaccccaaaaggtaccagtcacatgtgctgttggctgcgggtttttcggagccaggagactgtggtggaatcttgaggtgtgagcatggtgtcatcggcatagtgacaatgggtggcgaaggcgttgtcggtttcgccgatgtacgcgaccttctgtggctagaagatgatgccatggagcaaggggtcaaagattacgtggaacagctcggcaacgcctttgggtccggcttcaccaatcaaatttgcgagcaagtcaacctccttaaagagtcactcgttgggcaggactccatactagagaagtcgctcaaggccttagtgaagatcatatcagcactggttatcgtggtgagaaatcacgatgacctaatcacggtaacagccacactagccctcataggttgtacctcatcgccctggcggtggctcaagcaaaaagtgtcccaatactatgggatccctatggctgagaggcaaaataacaactggctaaagaaattcacagagatgaccaacgcctgcaagggtatggagtggatagctatgaaaatccaaaagtttattgaatggttaaaagtgaagatcttgccggaagtcagggagaaacatgagtttctaaatagattgaaacagctacccttgttggagagtcaaattgccactattgagcagagtgcaccatcacagagtgaccaagagcaattgttctccaatgtccagtactttgcacactactgcaggaagtatgccccactatacgctgctgaggctaagagagtcttctctttggagaagaaaatgagcaattacatacagttcaagtccaaatgccgtattgagcctgtctgtctgttgctccatggcagccccggagctggaaaatcagtagcaacaaacctaatcgggcgctcccttgctgaaaagctcaacagctcggtgtattccctaccaccggatcctgaccactttgatggctataaacagcaggccgttgtgatcatggatgatctttgccagaatccagatgggaaggatgtgtcactattttgccagatggtgtcaagtgtcgacttcgtcccacctatggcagcactagaagaaaagggcattctcttcacttcaccatttgttctcgcttccaccaatgcgggatccatcaatgctcccactgtgtcagacagcagggccttagccaggaggttccattttgacatgaacatcgaagtcatttccatgtatagccaaaacggtaagattaatatgccaatgtcagttaagatctgtgacgaggagtgctgcccggtcaattttaagaagtgttggccactcgtgtgtggcaaagccatccaattcattgatagaagaacacaggtgagatactcattagatatgcttgtgactgaaatgttcagagaatataatcataggcacagtgtgggagccacactcgaggcgcttttccaaggtccacctatttatagagagatcaagatcagcgtagcaccggaaaccccccccccaccatcaatagcagatctactgaagcctgtagatagggaagcagttagagagtactgtaaggaaaaagggtggatagtaccagagatcaactccaccctacagattgagaagcatgtgagcagggcttttatctgtctacaagcactcaccacatttgtctccgtggcggggataatatacatcatttacaaattgttcgctggcttccagggggcctacactggtatgcccaatccaaagcccaaagtgccaaccttaagacaggccaaagtgcagggaccagcatttgagtttgcagtagcaatgatgaagagaaacgctagcactgtgaaaactgagtatggcgagtttacaatgctcgggatctatgacaaatgggcagtgttgccccgtcatgccaaacctgggcccaccatattaatgaatgaccatgaagtgggtgtggtggacgctaaagagctagtggataaagatggcaccaacctggaactaacactcctgaagctcagcagaaatgaaaagtttagggacatccgaggctttctggcgagggaagaggtagaggttaatgaagccgtacttgctatcaacaccagcaagttcccaaacatgtacatcccagtgggacaggtgacagattatggttttctcaacctgggcgggacccccactaagcgcatgttaatgtataactttccaactcgtgcgggacagtgcggtggcgtcctaatgtcaacaggaaaggtccttggaatacatgtcggtgggaatgggcaccagggtttctcagcagccttgttgcgtcattatttcaatgatgagcagggtgagatcgagttcattgaaagctccaaggaagctggcttcccagtcatcaataccccaagcaaaacaaaactcgaacctagtgtcttccatcatgtatttgaaggggataaagagccggcagtgctgaggaacggagaccctaggctgaaggccaactttgaagaggccattttctccaagtacattggaaatgtgaacacacacgtagacgaatacatgatggaagctgtggaccattatgcaggacaattagcaactctggacatcagcacggaacccatgaggttggaggacgcagtttatggtactgagggcttagaggctctagatctgaacacaagtgcaggatacccctatgttgcccttggtattaaaaagagagatattctgtcaaagaagactaaggacctgacaaagctaaaagaatgcatggacaagtatggcttgaatctccctatggtcacctacgtgaaagatgaactgagatcagcagagaaggtggcaaagggaaaatctagactaattgaggcatctagtctcaatgactcagtggccatgaggcagacttttggaaatctgtacaaaactttccacctgaatccaggcatagtaacaggcagtgcggttgggtgtgaccccgatctcttctggagcaaaatccccgtcatgctcgatggtcacctcattgccttcgactacagtggttacgatgccagcttgagccctgtatggtttgcatgtctgaagttattgctggaaaagctaggatactcccacaaagaaaccaactacatagattacttatgcaactcccatcatttgtacagggataaacattactttgtgagaggaggtatgccatccgggtgctcagggaccagcatcttcaactcaatgatcaacaatattatcattagaacattaatgctgaaggtgtacaaaggaatagatttggaccaatttaggatgatagcatatggtgatgatgtcattgcatcatacccgcatcctattgacgctgccttgctagcagaggcaggaagagggtatggtctaatcatgacaccagccgataagggagaatgtttcaatgaagtgacatggaccaacgtcaccttcctgaaaaggtacttcagagcagatgagcaatacccctttctcatccaccctgtgatgccaatgaaggacatccatgaatcaatcaggtggaccaaagatcccagaaacacccaagaccatgtgcgatccttgtgtttgctggcctggcacaacggagagcaagaatatgaggaatttgtgtcgaaggtcaggagcgtccccgttgggcgctgcttagccttgcctgcgttttcgactctgcgtaggaaatggctggactccttttaacttagagtttactttgatttaattcaactgccttacccctaccacactcacccaactagtaagcggcgtggtaggctcaaattctccgcattcggtgacatcaacatctaagtcttttccatgtctagccaatacgctaacattactatgccaattctattactatctgcgacaaagac

SEQ ID NO: 2 - DNA, 26 bp, synthetic construct
  source 1..26: mol_type = other DNA
ggytayatncantgytggtaycarac

SEQ ID NO: 3 - DNA, 36 bp, synthetic construct
  source 1..36: mol_type = other DNA
ggtgctcactaggaggtcyctrttrtartcytcccr

SEQ ID NO: 4 - DNA, 20 bp, synthetic construct
  source 1..20: mol_type = other DNA
ttaaaacagcctgtgggttg

SEQ ID NO: 5 - DNA, 19 bp, synthetic construct
  source 1..19: mol_type = other DNA
gtaccagaagccgaaatgg

SEQ ID NO: 6 - DNA, 18 bp, synthetic construct
  source 1..18: mol_type = other DNA
ctagcactcagactagtc

SEQ ID NO: 7 - DNA, 17 bp, synthetic construct
  source 1..17: mol_type = other DNA
gatacgtggtggatgat

SEQ ID NO: 8 - DNA, 19 bp, synthetic construct
  source 1..19: mol_type = other DNA
ggcctctagcaattgtgtc

SEQ ID NO: 9 - DNA, 19 bp, synthetic construct
  source 1..19: mol_type = other DNA
tatatggttacaactcact

SEQ ID NO: 10 - DNA, 18 bp, synthetic construct
  source 1..18: mol_type = other DNA
tgtggctagaagatgatg

SEQ ID NO: 11 - DNA, 18 bp, synthetic construct
  source 1..18: mol_type = other DNA
agctcaacagctcggtgt

SEQ ID NO: 12 - DNA, 18 bp, synthetic construct
  source 1..18: mol_type = other DNA
ggagctttcaatgaactc

SEQ ID NO: 13 - DNA, 18 bp, synthetic construct
  source 1..18: mol_type = other DNA
acctgaatccaggcatag

SEQ ID NO: 14 - DNA, 18 bp, synthetic construct
  source 1..18: mol_type = other DNA
ttgtgggagtatcctagc

SEQ ID NO: 15 - DNA, 18 bp, synthetic construct
  source 1..18: mol_type = other DNA
gaatcaatcaggtggacc

SEQ ID NO: 16 - DNA, 20 bp, synthetic construct
  source 1..20: mol_type = other DNA
taatgtgtcttgtgtcggct

SEQ ID NO: 17 - DNA, 20 bp, synthetic construct
  source 1..20: mol_type = other DNA
ttgccaatcaaaacttgttg

SEQ ID NO: 18 - DNA, 20 bp, synthetic construct
  source 1..20: mol_type = other DNA
caccgaatgcggagaattta

SEQ ID NO: 19 - DNA, 21 bp, synthetic construct
  source 1..21: mol_type = other DNA
tgtcctgtttccactgctgtt

SEQ ID NO: 20 - DNA, 20 bp, synthetic construct
  source 1..20: mol_type = other DNA
gaagaatcagtggagcgtgc

SEQ ID NO: 21 - DNA, 14 bp, synthetic construct
  source 1..14: mol_type = other DNA
ctctgaacaaatcc